(19) United States
(12) Patent Application Publication
(10) Pub. No.: US 2008/0208206 A1
(43) Pub. Date: Aug. 28, 2008

(54) Surgical tongs

(76) Inventor: Ignacio J. Calvo, Miami, FL (US)

Correspondence Address: RUBEN ALCOBA, ESQ., 3399 NW 72 AVENUE, SUITE211, MIAMI, FL, 33122, US

(21) Appl. No.: 11/709,532

(22) Filed: Feb. 22, 2007

(51) Int. Cl. A61B 17/58

(52) U.S. Cl. 606/105

(57) ABSTRACT

A modified surgical tongs for providing a surgical doctor with a tongs that will permit optimal fixation of the malleous to the tibia, of the olecranon, or of transverse fractures of the patella. The tongs comprises of a pair of hinged legs. Each hinged leg has a first and a second end, the first end of each hinged leg having a handle and the second end having at least one hook. The first hinged leg's perpendicular hook points approximately perpendicular to the plane of the handles. The second hinged leg's parallel hook points toward the perpendicular hook and is in a plane that is approximately parallel to the plane of the handles. Each hinged leg further defines an arcuate locking mechanism that is adjacent to each handle, and each arcuate locking mechanism is in the same plane as each handle.

### BACKGROUND

The present invention is a modified surgical tongs used to maintain anatomic reduction of fractures of medial malleolus, some fractures of olecranon, or patella while internal fixation (with canulated screws) is surgically performed. A tongs is an instrument for grasping and holding, consisting of two arms joined by a hinge or pivot.

Fractures of the medial malleolus are usually reduced and fixed to the proximal surface of the fracture at the distal epiphysis of the tibia with canulated screws that are inserted after the overdrilling of the malleolar fragment using a thin K wire as a guide. If the fracture is anatomically reduced and held with an appropriate clamp, it becomes very easy to optimally position the K guide wire over which the canulated screw is inserted.

The present method of performing anatomic reduction is by having a helper anatomically reduce the fracture, then having the surgeon insert the guide wire over which the canulated screw is inserted.

An objective of the present invention is to provide a surgical doctor with a tongs that will permit optimal fixation of the malleolus to the tibia.

A further objective of the present invention is to provide a surgical doctor with a tongs that will permit optimal fixation of a fractured olecranon.

Another objective of the present invention is to provide a surgical doctor with a tongs that will permit optimal fixation of transverse fractures of the patella.

### SUMMARY

The present invention is directed to a modified surgical tongs that satisfies the need for providing a surgical doctor with a tongs that will permit optimal fixation of the malleous to the tibia, of the olecranon, or of transverse fractures of the patella. The tongs comprises of a pair of hinged legs. Each hinged leg has a first and a second end, the first end of each hinged leg having a handle and the second end having at least one hook. The first hinged leg's perpendicular hook points approximately perpendicular to the plane of the handles. The second hinged leg's parallel hook points toward the perpendicular hook and is in a plane that is approximately parallel to the plane of the handles. Each hinged leg further defines an arcuate locking mechanism that is adjacent to each handle, and each arcuate locking mechanism is in the same plane as each handle.

In the preferred embodiment of the present invention, there is a separation of at least 3.5 centimeters between the tips of the hooks when the tongs is in a closed position, and each arcuate locking mechanism has a length of at least 4 centimeters.

In another embodiment of the present invention, the second hinged leg's second end has two parallel hooks that point toward the perpendicular hook and are in a plane that is approximately parallel to the plane of the handle, the parallel hooks flow from the end of the second end of the second hinged leg in an arcuate manner that is in a plane that is perpendicular to the plane of the handles.

### DRAWINGS

These and other features, aspects, and advantages of the present invention will become better understood with regard to the following description, appended claims, and drawings where:

FIG. 1 shows a side view of the surgical tongs of the first embodiment of the present invention;

FIG. 2 shows a bottom side view of the first embodiment of the present invention showing a perpendicular hook flowing outward toward the viewer and the parallel hook pointing toward the perpendicular hook;

FIG. 3 shows a bottom side view of the first embodiment of the present invention showing the invention in the open position;

FIG. 4 shows a view of the first embodiment of the present invention wherein one sees from the hinged legs first end to the second;

FIG. 5 shows how the first embodiment of the present invention is placed on a fracture;

FIG. 6 shows how the first embodiment of the present invention anatomically reduces the fracture;

FIG. 7 shows a side view of the surgical tongs of the second embodiment of the present invention;

FIG. 8 shows a bottom side view of the second embodiment of the present invention showing a perpendicular hook flowing outward toward the viewer and the parallel hook pointing toward the perpendicular hook;

FIG. 9 shows a bottom side view of the second embodiment of the present invention showing the invention in the open position;

FIG. 10 shows a view of the second embodiment of the present invention wherein one sees from the hinged legs first end to the second;

FIG. 11 shows how the second embodiment of the present invention is placed on a fracture;

FIG. 12 shows how the second embodiment of the present invention anatomically reduces the fracture;

FIG. 13 shows a side view of the surgical tongs of the third embodiment of the present invention;

FIG. 14 shows a bottom side view of the third embodiment of the present invention showing a perpendicular hook flowing outward toward the viewer and two parallel hooks pointing toward the perpendicular hook;

FIG. 15 shows a bottom side view of the third embodiment of the present invention showing the invention in the open position;

FIG. 16 shows a view of the third embodiment of the present invention wherein one sees from the hinged legs first end to the second;

FIG. 17 shows how the third embodiment of the present invention is placed on a fracture;

FIG. 18 shows how the third embodiment of the present invention anatomically reduces the fracture;

FIG. 19 shows a side view of the surgical tongs of the fourth embodiment of the present invention;

FIG. 20 shows a bottom side view of the fourth embodiment of the present invention showing a perpendicular hook flowing outward toward the viewer and two parallel hooks pointing toward the perpendicular hook;

FIG. 21 shows a bottom side view of the fourth embodiment of the present invention showing the invention in the open position;

FIG. 22 shows a view of the fourth embodiment of the present invention wherein one sees from the hinged legs first end to the second;

FIG. 23 shows how the fourth embodiment of the present invention is placed on a fracture; and

FIG. 24 shows how the fourth embodiment of the present invention anatomically reduces the fracture.

### DESCRIPTION

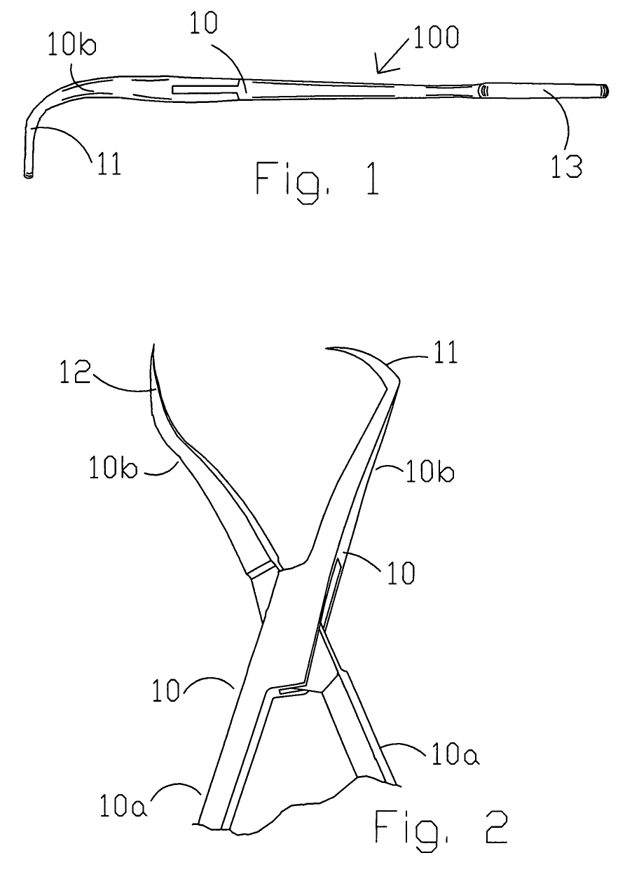

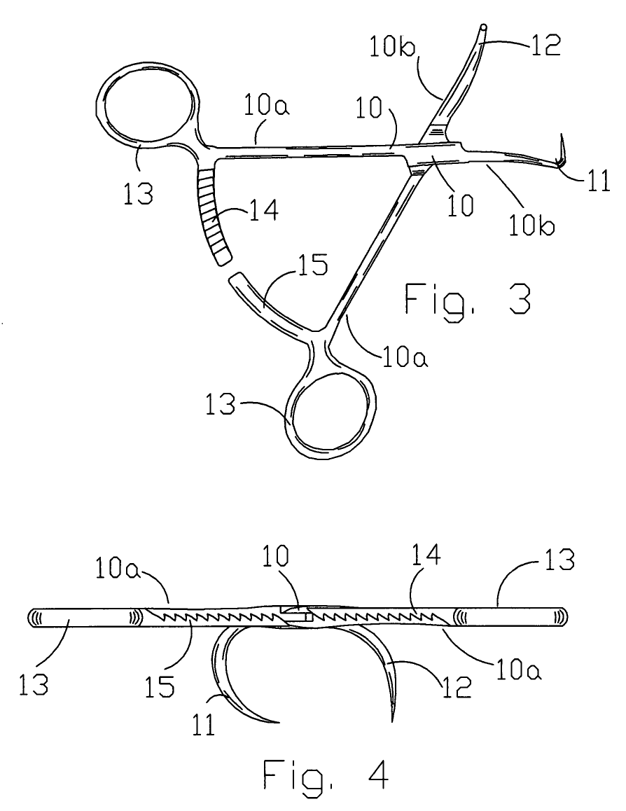

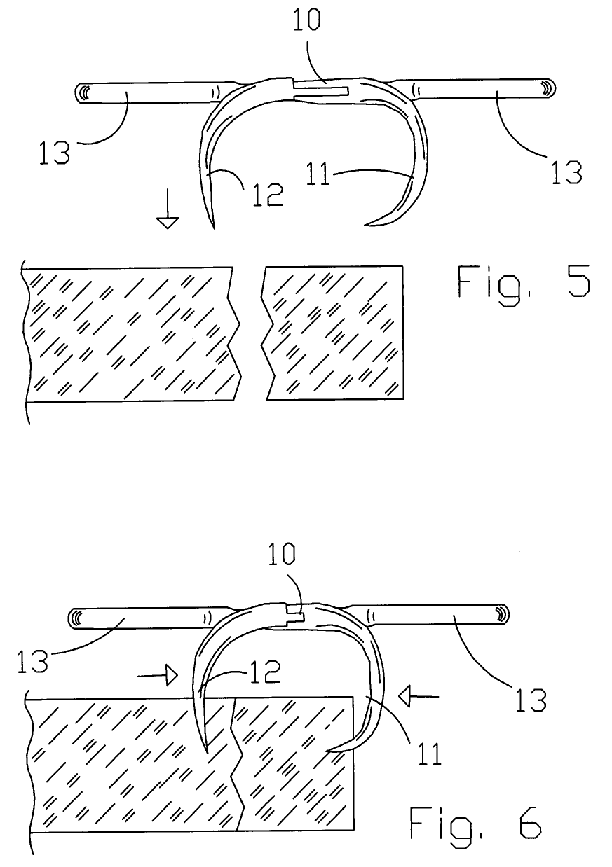

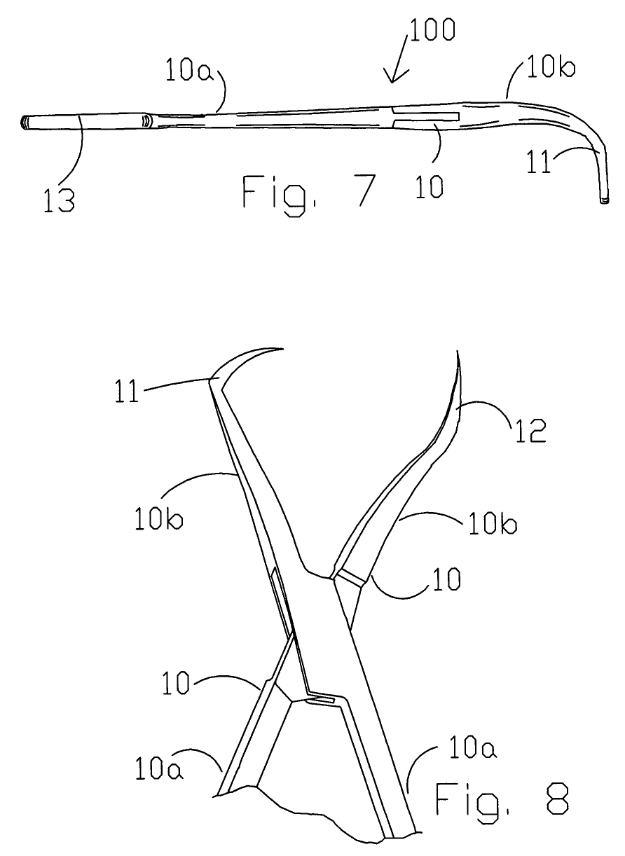

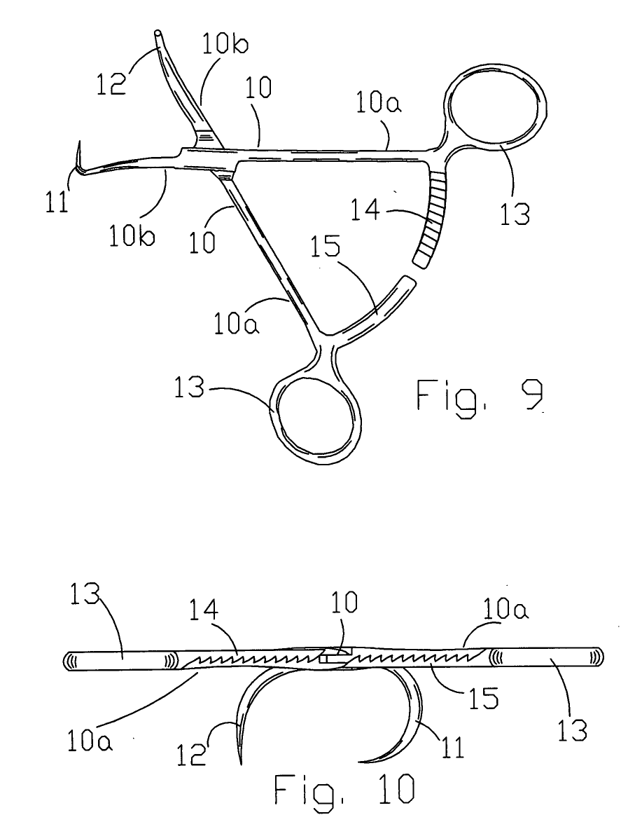

As shown in FIGS. 1-4 and 7-10, a surgical tongs 100 for reducing anatomical fractures comprises a pair of hinged legs 10, each hinged legs 10 having a first 10a and a second end 10b, the first end 10a of each hinged leg 10 having a handle 13 and the second end 10b having at least one hook 11-12, the first hinged leg's 10 hook 12 points approximately perpendicular to the plane of the handles 13 and will be called the perpendicular hook 12 hereinafter, and the second hinged leg's 10 hook 11 points toward the perpendicular hook 12 and is in a plane that is approximately parallel to the plane of the handles 13 and will be called the parallel hook 11 hereinafter, each hinged leg 10 further defines an arcuate locking mechanism 15-16 that is adjacent to each handle 13, and each arcuate locking mechanism 15-16 is in the same plane as each handle 13.

In the preferred embodiment of the present invention, there is a separation of at least 3.5 centimeters between the tips of the hooks 11-12 when the tongs 100 is in a closed position, and each arcuate locking mechanism 14-15 has a length of at least 4 centimeters.

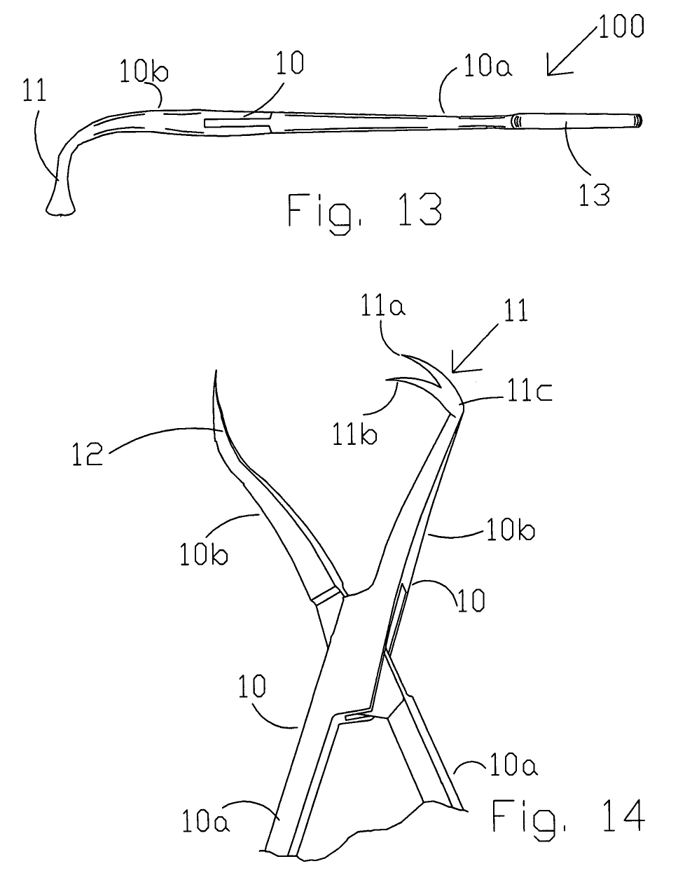

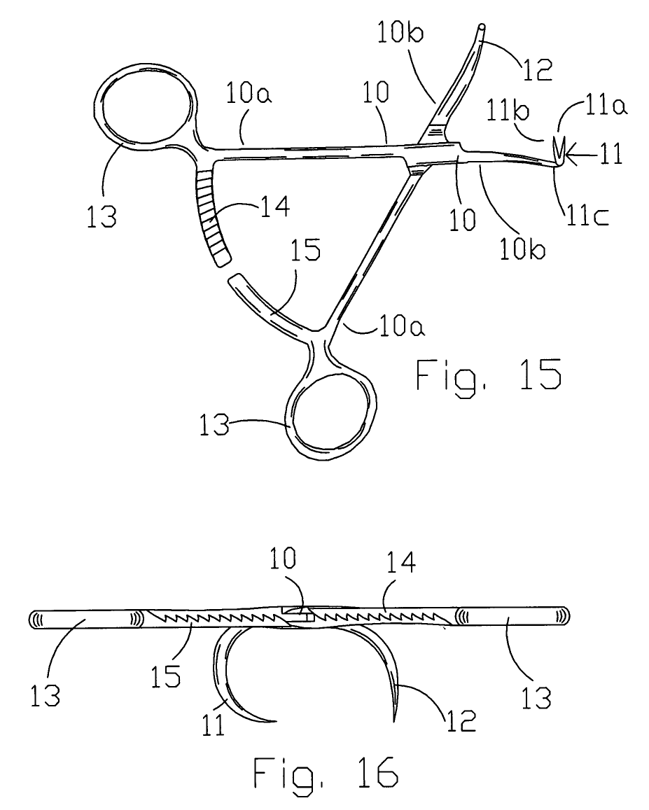

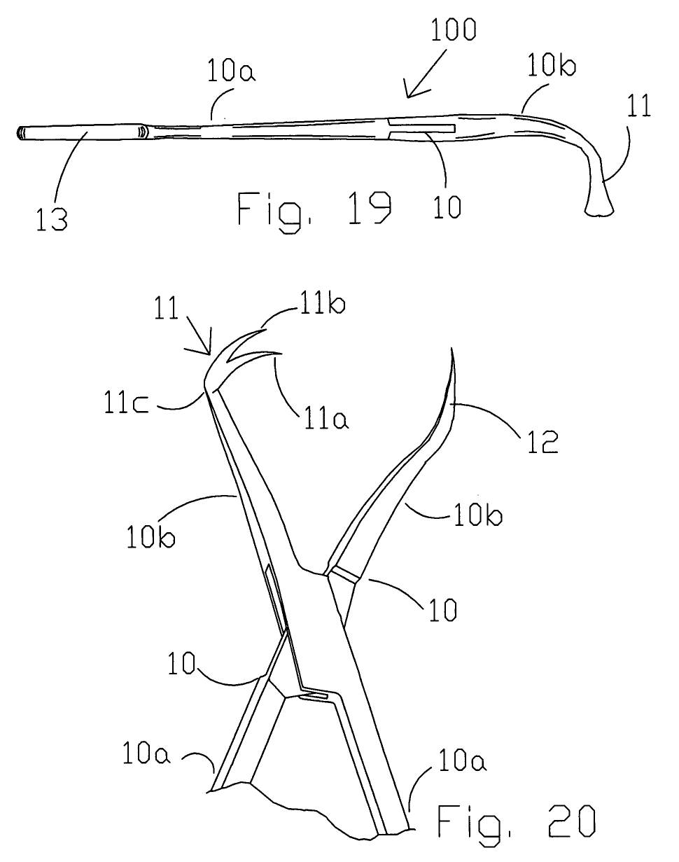

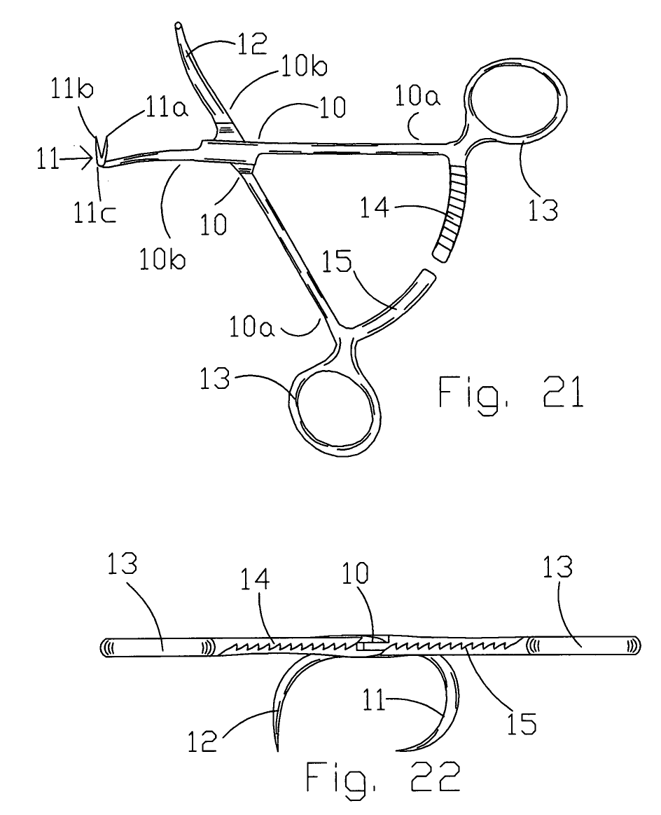

In another embodiment of the present invention, As seen in FIGS. 13-15, and 19-21 the second hinged leg's second end has two parallel hooks 11a-11b that point toward the perpendicular hook 12 and are in a plane that is approximately parallel to the plane of the handle 13, the parallel hooks 11a-11b flow from the end of the second end 10b of the second hinged leg 10 in an arcuate manner 11c that is in a plane that is perpendicular to the plane of the handles 13. In a preferred embodiment, the parallel hooks 11a-11b are at least 1 centimeter apart.

The surgical tongs 100 is made of materials known in the art of surgical tools. The tongs 10 can be made to accommodate right and left handed physicians or to facilitate working on the right or left tibia malleolar fractures.

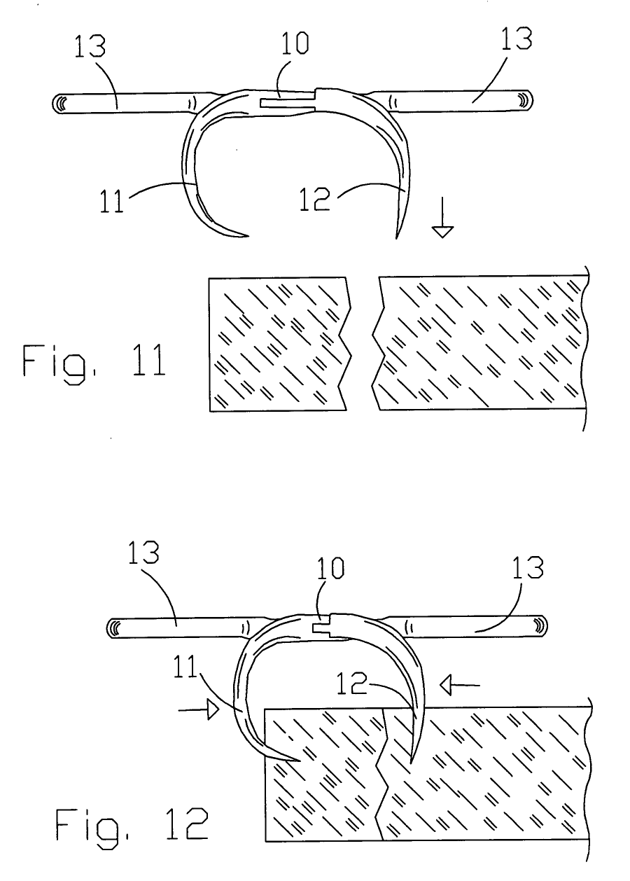

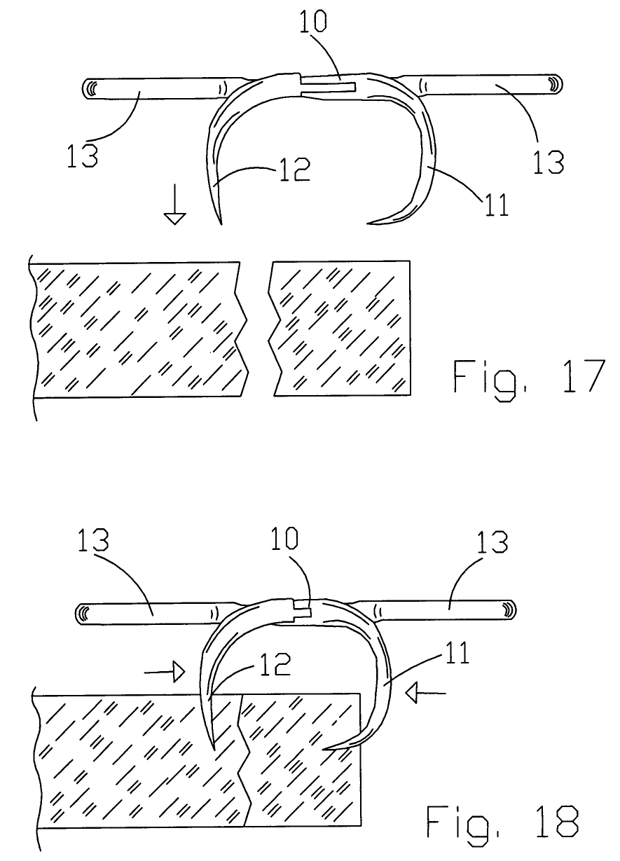

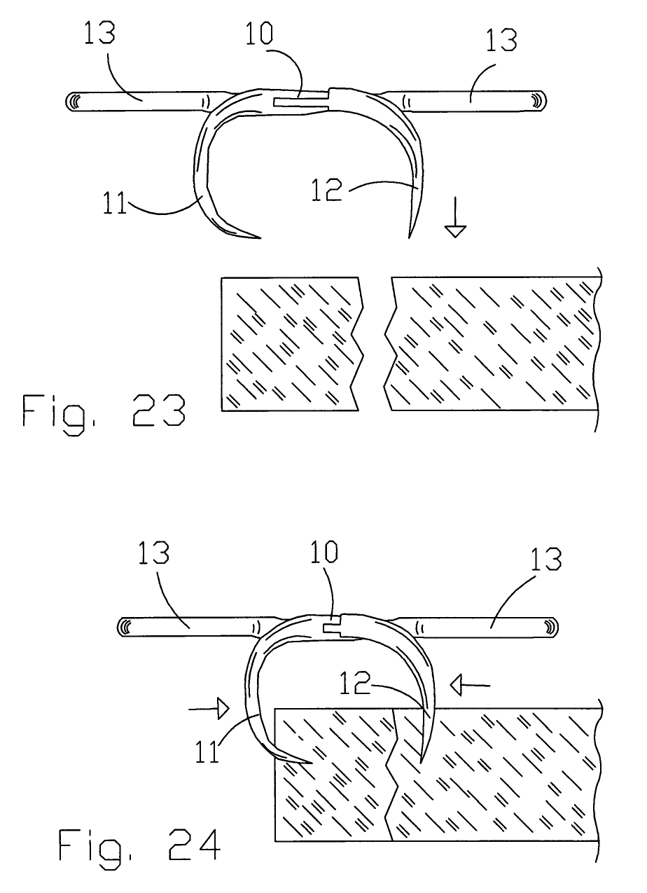

Fractures of the medial malleolus are usually reduced and fixed to the proximal surface of the fracture at the distal epiphysis of the tibia with canualted screws that are inserted after overdrilling of the malleolar fragment using a thin K wire as a guide. As seen in FIGS. 5-6, 11-12, 17-18, and 23-24, the surgical tongs 100, when anatomically reducing the fracture of the medial malleolus to the tibia, is used by inserting the perpendicular hook 12 through a small orifice at the proximal component of the fracture at the tibia and holding the distal fragment (malleolus) with the parallel hook 11 of the tongs 100.

The same fixation principles can be applied to fixing a fractured olecranon or in fixing transverse fractures of the patella.

An advantage of the present invention is that it provides a surgical doctor with a tongs that permits optimal fixation of the malleolus to the tibia.

A further advantage of the present invention is that it provides a surgical doctor with a tongs that permits optimal fixation of a fractured olecranon.

Another advantage of the present invention is that it provides a surgical doctor with a tongs that permits optimal fixation of transverse fractures of the patella.

What is claimed is:

1. A surgical tongs for reducing anatomical fractures comprises a pair of hinged legs, each hinged legs having a first and a second end, the first end of each hinged leg having a handle and the second end having at least one hook, the first hinged leg's hook points approximately perpendicular to the plane of the handles and will be called the perpendicular hook hereinafter, and the second hinged leg's hook points toward the perpendicular hook and is in a plane that is approximately parallel to the plane of the handles and will be called the parallel hook hereinafter, each hinged leg further defines an arcuate locking mechanism that is adjacent to each handle, and each arcuate locking mechanism is in the same plane as each handle.

2. The surgical tongs of claim 1, wherein there is a separation of at least 3.5 centimeters between the tips of the hooks when the tongs is in a closed position, and each arcuate locking mechanism has a length of at least 4 centimeters.

3. The surgical tongs of claim 1, wherein the second hinged leg's second end comprises of two parallel hooks that point toward the perpendicular hook and are in a plane that is approximately parallel to the plane of the handle, the parallel hooks flow from the end of the second end of the second hinged leg in an arcuate manner that is in a plane that is perpendicular to the plane of the handles.

4. The surgical tongs of claim 3, wherein the parallel hooks are at least 1 centimeter apart.

5. The surgical tongs of claim 4, wherein there is a separation of at least 3.5 centimeters between the tips of the hooks when the tongs is in a closed position, and each arcuate locking mechanism has a length of at least 4 centimeters.
